(19) United States
(12) Patent Application Publication
(10) Pub. No.: US 2014/0200466 A1
(43) Pub. Date: Jul. 17, 2014

(54) POLYMERIC MATERIALS

(71) Applicants: Nuno Sereno, Merseyside (GB); Vaughn Williams, Lancashire (GB); Marcus Jarman-Smith, Lancashire (GB); Nikolaj Gadegaard, Glasgow (GB)

(72) Inventors: Nuno Sereno, Merseyside (GB); Vaughn Williams, Lancashire (GB); Marcus Jarman-Smith, Lancashire (GB); Nikolaj Gadegaard, Glasgow (GB)

(73) Assignee: INVIBIO LIMITED, Lancashire (GB)

(21) Appl. No.: 14/128,686

(22) PCT Filed: Jun. 21, 2012
(86) PCT No.: PCT/GB2012/051434

§ 371 Date: Mar. 28, 2014

(30) Foreign Application Priority Data
Jun. 24, 2011 (GB) 1110729.9

(51) Int. Cl. A61L 31/06; A61N 1/36; A61B 5/00

(52) U.S. Cl. CPC A61L 31/06; A61B 5/6869; A61N 1/36
USPC 600/508; 607/116; 264/328.1; 428/156

(57) ABSTRACT

Topographical features, such as projections or recesses, having a maximum dimension which is less than 3 μm, with the features being separated by a distance which is less than 10 μm are transferred to polyetheretherketone, on an industrial scale, by injection moulding relatively low viscosity PEEK, using a mould in which is arranged a master structure which carries the desired topography. The topographical features increase the water contact angle of a surface which includes them and such a modified surface has been shown to influence cell attachment and differentiation. Parts which incorporate the topographical features may be used in medical devices such as implantable medical devices for cardiology or for neuromodulation.

This invention relates to polymeric materials and particularly, although not exclusively, relates to polyaryletherketones, for example polyetheretherketone (PEEK).

PEEK is a well-known high performance thermoplastic which is used in wide-ranging industrial and medical applications. However, in some cases, parts made from PEEK are more hydrophilic or more hydrophobic than desirable. For example, if it is desired to coat PEEK with a relatively hydrophobic coating material, it could be desirable for the PEEK to be more hydrophobic than it naturally is. It is an object of the present invention to address this problem.

Implantable medical devices, such as for cardiology or neuromodulation, can advantageously be made from PEEK. However, a significant disadvantage of the use of PEEK is that, over time, water may migrate through the PEEK into an internal region of the device. This is undesirable. It is an object of the present invention to address this problem.

According to a first aspect of the invention, there is provided a part which comprises a polymeric material, wherein a surface of said part includes an array of topographical features which comprise spaced apart projections or recesses, wherein said topographical features have a maximum dimension which is less than 3 μm and said topographical features are separated by a distance which is less than 10 μm, wherein said polymeric material has a moiety of formula

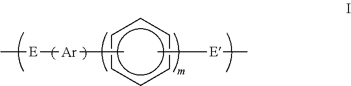

and/or a moiety of formula

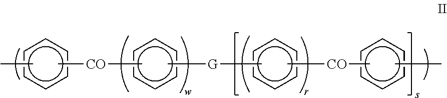

and/or a moiety of formula

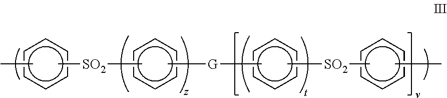

wherein m, r, s, t, v, w and z independently represent zero or a positive integer, E and E′ independently represent an oxygen or a sulphur atom or a direct link, G represents an oxygen or sulphur atom, a direct link or a —O-Ph-O— moiety where Ph represents a phenyl group and Ar is selected from one of the following moieties (i)**, (i) to (iv) which is bonded via one or more of its phenyl moieties to adjacent moieties

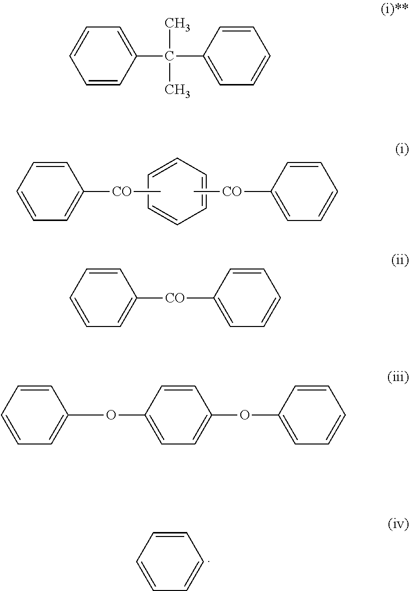

Advantageously, the provision of said topographical features is found to influence the hydrophobicity and/or hydrophilicity of the surface; and by selection of appropriate topographical features, the hydrophobicity and/or hydrophilicity can be controlled in a reproducible and predictable manner. The topographical features may be used to enhance bonding of coatings to polymeric materials such as PEEK. Also, in situations wherein the polymeric material is in contact in use with a fluid, for example water, the surface of the material may be provided with topographical features to make it less likely to absorb the fluid. Said array of topographical features may include at least 100, preferably at least 1000, more preferably at least 10000 of said topographical features. Said array suitably includes at least 100,000 of said topographical features per mm², preferably at least 3,000,000 per mm², more preferably at least 6,000,000 per mm².

Said part suitably includes at least 10⁷, preferably at least 10⁸, more preferably at least 10⁹ of said topographical features. Said part suitably includes at least 10⁷, preferably at least 10⁸, more preferably at least 10⁹ projections.

When said topographical features comprise projections, the maximum height of said projections may be less than 10 μm, preferably less than 500 nm, more preferably less than 200 nm. The projections may have a height of at least 10 nm.

When said topographical features comprise recesses, the maximum depth of the recesses may be less than 10 μm, preferably less than 500 nm, more preferably less than 200 nm. The recesses may have a depth of at least 10 nm.

Said topographical features preferably comprise projections or recesses, but not both projections and recesses. Preferably, said topographical features comprise projections, preferably solely projections.

Said topographical features may be circular, triangular or square in plan view. Preferably, said topographical features are circular in plan view.

Suitably, at least 50%, preferably at least 90%, more preferably substantially all of the topographical features (e.g. projections) associated with said surface have maximum dimensions as specified herein. Said surface preferably includes less than 10% (preferably substantially 0%) of topographical features (e.g. projections) which have a maximum dimension greater than 3 μm.

Said topographical features (e.g. projections) may have a maximum dimension (e.g. diameter in the case of a circular projections) of less than 0.5 μm, preferably less than 0.25 μm, more preferably less than 0.2 μm.

Said topographical features may be spaced apart (i.e. the shortest distance between edges of adjacent features) by a distance of less than 5 μm, preferably less than 1 μm, more preferably less than 750 nm, especially less than 500 nm.

Said topographical features may individually have a maximum area of less than 20 μm², suitably less than 0.8 μm², preferably less than 0.2 μm², especially less than 0.14 μm². Preferably, at least 50%, 80%, 90%, 95% or 99% of topographical features provided on said surface have the aforementioned maximum areas. The minimum area of said topographical features (preferably at least 50%, 80%, 90%, 95% or 99% of the topographical features provided on said surface) may be at least 200 nm² or at least 7000 nm².

Said array of topographical features preferably comprises at least 1000, preferably at least 10,000, especially at least 10⁶ topographical features which have substantially the same maximum dimension (e.g. diameter). Said array may comprise at least 1000, preferably at least 10,000, especially at least 10⁶ topographical features which have substantially the same height, or in the case of recesses, substantially the same depth. Said array of topographical features may comprise at least 1000, preferably at least 10,000, especially at least 10⁶ topographical features of substantially the same surface area. Said array of topographical features preferably comprises at least 1000, preferably at least 10,000, especially at least 10⁶ topographical features (preferably projections) of substantially the same size and substantially the same shape.

The device suitably has an arrangement of topographical features arrayed in a pattern based on a notional symmetrical lattice in which the distance between nearest neighbour notional lattice points is C and is between 10 nm and 10 μm, and wherein the topographical features are locally mis-ordered such that the centre of each topographical feature is a distance of up to one half of C from its respective notional lattice point.

Preferably, C is at least 20 nm, at least 30 nm, at least 40 nm, at least 50 nm, at least 60 nm, at least 70 nm, at least 80 nm, at least 90 nm, at least 100 nm, at least 110 nm, at least 120 nm, at least 130 nm, at least 140 nm, at least 150 nm, at least 160 nm, at least 170 nm, at least 180 nm, at least 190 nm, at least 200 nm, at least 210 nm, at least 220 nm, at least 230 nm, at least 240 nm, at least 250 nm, at least 260 nm, at least 270 nm, at least 280 nm, at least 290 nm or about 300 nm.

Preferably, C is at most 9 μm, at most 8 μm, at most 7 μm, at most 6 μm, at most 5 μm, at most 4 μm, at most 3 μm, at most 2 μm, at most 1 μm, at most 900 nm, at most 800 nm, at most 700 nm, at most 600 nm, at most 500 nm, at most 400 nm.

The most preferred range for C is between 30 nm and 3 μm.

Preferably, the height or depth (e.g. the average height or depth) of the topographical features is at least 5%, more preferably at least 10%, of C from the remainder of the surface of the device. For example, the height or depth of the topographical features may be at least 10 nm.

Preferably, each topographical feature has the same shape. The topographical features may be cylindrical pits or projections, cuboid pits or projections, hemi-spherical pits or projections, part-spherical pits or projections, or another regular shape.

Preferably, the diameter of the topographical features is at least 10%, more preferably at least 20%, at least 30%, at least 40% or at least 50%, of C. For example, the diameter of the topographical features may be at least 20 nm.

Preferably, the centre of each topographical feature is at most 45%, more preferably at most 40%, at most 35%, at most one third, at most 30%, at most 25%, at most 20%, at most 15%, at most 10% or at most 5%, of C from its respective notional lattice point.

Preferably, for at least 50% of the topographical features, the centre of each topographical feature is between one tenth and one quarter of C from its respective notional lattice point. More preferably, at least 60%, at least 70%, at least 80% or at least 90% of the topographical features satisfy this criterion. The lower limit for the distance of the centre of each topographical feature from its respective notional lattice point is preferably at least 12% of C, at least 14% of C or at least 16% of C. The upper limit for the distance of the centre of each topographical feature from its respective notional lattice point is preferably at most 22% of C, at most 20% of C or at least 18% of C.

The nature of the symmetry on which the notional lattice is based may be selected from a parallelogram lattice, a rectangular lattice, a square lattice, a rhombic lattice, a trigonal lattice and a hexagonal lattice. Preferably, the notional lattice is either a rectangular lattice or a square lattice.

In one embodiment, topographical features may be defined to mimic the Lotus effect exhibited by the leaves of the lotus flower.

Said part may comprise a body which has a first side and a second side, wherein said surface which includes said array is provided at said first side and wherein said polymeric material defines the first side, second side and said array of topographical features. Said projections are suitably defined by said polymeric material. Preferably, said polymeric material is substantially homogenously distributed within said body and/or the same composition which comprises or consists essentially of said polymeric material is present at said first side and second side of the body and in projections which define said topographical features.

Unless otherwise stated in this specification, a phenyl moiety has 1,4-, linkages to moieties to which it is bonded.

In (i), the middle phenyl may be 1,4- or 1,3-substituted. It is preferably 1,4-substituted.

Said polymeric material may include more than one different type of repeat unit of formula I; and more than one different type of repeat unit of formula II; and more than one different type of repeat unit of formula III. Preferably, however, only one type of repeat unit of formula I, II and/or III is provided.

Said moieties I, II and III are suitably repeat units. In the polymeric material, units I, II and/or III are suitably bonded to one another—that is, with no other atoms or groups being bonded between units I, II and III.

Phenyl moieties in units I, II and III are preferably not substituted. Said phenyl moieties are preferably not cross-linked.

Where w and/or z is/are greater than zero, the respective phenylene moieties may independently have 1,4- or 1,3-linkages to the other moieties in the repeat units of formulae II and/or III. Preferably, said phenylene moieties have 1,4-linkages.

Preferably, the polymeric chain of the polymeric material does not include a —S— moiety. Preferably, G represents a direct link.

Suitably, “a” represents the mole % of units of formula I in said polymeric material, suitably wherein each unit I is the same; “b” represents the mole % of units of formula II in said polymeric material, suitably wherein each unit II is the same; and “c” represents the mole % of units of formula III in said polymeric material, suitably wherein each unit III is the same. Preferably, a is in the range 45-100, more preferably in the range 45-55, especially in the range 48-52. Preferably, the sum of b and c is in the range 0-55, more preferably in the range 45-55, especially in the range 48-52. Preferably, the ratio of a to the sum of b and c is in the range 0.9 to 1.1 and, more preferably, is about 1. Suitably, the sum of a, b and c is at least 90, preferably at least 95, more preferably at least 99, especially about 100. Preferably, said polymeric material consists essentially of moieties I, II and/or III.

Said polymeric material may be a homopolymer having a repeat unit of general formula

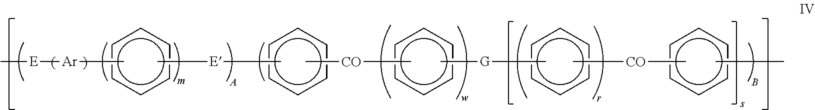

or a homopolymer having a repeat unit of general formula

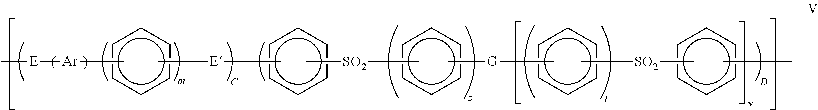

or a random or block copolymer of at least two different units of IV and/or V, wherein A, B, C and D independently represent 0 or 1 and E, E′, G, Ar, m, r, s, t, v, w and z are as described in any statement herein.

Preferably, m is in the range 0-3, more preferably 0-2, especially 0-1. Preferably, r is in the range 0-3, more preferably 0-2, especially 0-1. Preferably t is in the range 0-3, more preferably 0-2, especially 0-1. Preferably, s is 0 or 1. Preferably v is 0 or 1. Preferably, w is 0 or 1. Preferably z is 0 or 1.

Suitably, at least one of A or B is 1. Preferably, A is 1. Preferably A and B are 1. Suitably, at least one of C or D is 1. Preferably, C is 1. Preferably, C and D are 1.

Preferably, said polymeric material is a homopolymer having a repeat unit of general formula IV.

Preferably Ar is selected from the following moieties (xi)** and (vii) to (x)

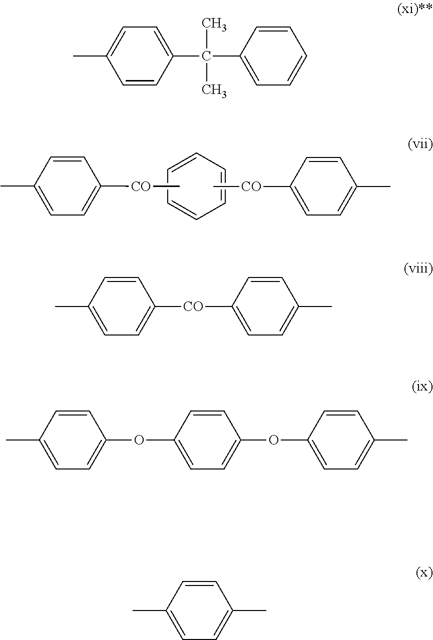

In (vii), the middle phenyl may be 1,4- or 1,3-substituted. It is preferably 1,4-substituted.

Suitable moieties Ar are moieties (i), (ii), (iii) and (iv) and, of these, moieties (i), (ii) and (iv) are preferred. Other preferred moieties Ar are moieties (vii), (viii), (ix) and (x) and, of these, moieties (vii), (viii) and (x) are especially preferred.

An especially preferred class of polymeric materials are polymers (or copolymers) which consist essentially of phenyl moieties in conjunction with ketone and/or ether moieties. That is, in the preferred class, said polymeric material does not include repeat units which include —S—, —SO₂— or aromatic groups other than phenyl. Preferred polymeric materials of the type described include:

(a) a polymeric material consisting essentially of units of formula IV wherein Ar represents moiety (iv), E and E′ represent oxygen atoms, m represents 0, w represents 1, G represents a direct link, s represents 0, and A and B represent 1 (i.e. polyetheretherketone).

(b) a polymeric material consisting essentially of units of formula IV wherein E represents an oxygen atom, E′ represents a direct link, Ar represents a moiety of structure (i), m represents 0, A represents 1, B represents 0 (i.e. polyetherketone);

(c) a polymeric material consisting essentially of units of formula IV wherein E represents an oxygen atom, Ar represents moiety (i), m represents 0, E′ represents a direct link, A represents 1, B represents 0, (i.e. polyetherketoneketone).

(d) a polymeric material consisting essentially of units of formula IV wherein Ar represents moiety (i), E and E′ represent oxygen atoms, G represents a direct link, m represents 0, w represents 1, r represents 0, s represents 1 and A and B represent 1. (i.e. polyetherketoneetherketoneketone).

(e) a polymeric material consisting essentially of units of formula IV, wherein Ar represents moiety (iv), E and E′ represents oxygen atoms, G represents a direct link, m represents 0, w represents 0, s, r, A and B represent 1 (i.e. polyetheretherketoneketone).

(f) a polymeric material comprising units of formula IV, wherein Ar represents moiety (iv), E and E′ represent oxygen atoms, m represents 1, w represents 1, A represents 1, B represents 1, r and s represent 0 and G represents a direct link (i.e. polyether-diphenyl-ether-phenyl-ketone-phenyl-).

Said polymeric material may be amorphous or semi-crystalline. Said polymeric material is preferably semi-crystalline. The level and extent of crystallinity in a polymer is preferably measured by wide angle X-ray diffraction (also referred to as Wide Angle X-ray Scattering or WAXS), for example as described by Blundell and Osborn (Polymer 24, 953, 1983). Alternatively, crystallinity may be assessed by Differential Scanning calorimetry (DSC).

The level of crystallinity in said polymeric material may be at least 1%, suitably at least 3%, preferably at least 5% and more preferably at least 10%. In especially preferred embodiments, the crystallinity may be greater than 30%, more preferably greater than 40%, especially greater than 45%.

The main peak of the melting endotherm (Tm) for said polymeric material (if crystalline) may be at least 300° C.

Said polymeric material may consist essentially of one of units (a) to (f) defined above.

Said polymeric material preferably comprises, more preferably consists essentially of, a repeat unit of formula (XX)

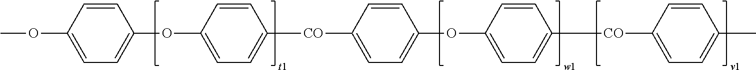

where t1, and w1 independently represent 0 or 1 and v1 represents 0, 1 or 2. Preferred polymeric materials have a said repeat unit wherein t1=1, v1=0 and w1=0; t1=0, v1=0 and w1=0; t1=0, w1=1, v1=2; or t1=0, v1=1 and w1=0. More preferred have t1=1, v1=0 and w1=0; or t1=0, v1=0 and w1=0. The most preferred has t1=1, v1=0 and w1=0.

In preferred embodiments, said polymeric material is selected from polyetheretherketone, polyetherketone, polyetherketoneetherketoneketone and polyetherketoneketone. In a more preferred embodiment, said polymeric material is selected from polyetherketone and polyetheretherketone. In an especially preferred embodiment, said polymeric material is polyetheretherketone.

Said polymeric material suitably has a melt viscosity (MV) of at least 0.06 kNsm⁻², preferably has a MV of at least 0.085 kNsm⁻², more preferably at least 0.12 kNsm⁻², especially at least 0.14 kNsm⁻².

MV is suitably measured using capillary rheometry operating at 400° C. at a shear rate of 1000 s⁻¹ using a tungsten carbide die, 0.5 mm×3.175 mm.

Said polymeric material may have a MV of less than 1.00 kNsm⁻², suitably less than 0.5 kNsm⁻², preferably less than 0.4 kNsm⁻², more preferably less than 0.3 kNsm⁻², especially less than 0.25 kNsm⁻², or even less than 0.2 kNsm⁻².

Said polymeric material may have a MV in the range 0.09 to 0.4 kNsm⁻², preferably in the range 0.09 to 0.3 kNsm⁻², more preferably in the range 0.09 to 0.25 kNsm⁻², especially in the range 0.09 to 0.20 kNsm⁻².

Said polymeric material may have a tensile strength, measured in accordance with ISO527 (specimen type 1b) tested at 23° C. at a rate of 50 mm/minute of at least 20 MPa, preferably at least 60 MPa, more preferably at least 80 MPa. The tensile strength is preferably in the range 80-110 MPa, more preferably in the range 80-100 MPa.

Said polymeric material may have a flexural strength, measured in accordance with ISO178 (80 mm×10 mm×4 mm specimen, tested in three-point-bend at 23° C. at a rate of 2 mm/minute) of at least 50 MPa, preferably at least 100 MPa, more preferably at least 145 MPa. The flexural strength is preferably in the range 145-180 MPa, more preferably in the range 145-164 MPa.

Said polymeric material may have a flexural modulus, measured in accordance with ISO178 (80 mm×10 mm×4 mm specimen, tested in three-point-bend at 23° C. at a rate of 2 mm/minute) of at least 1 GPa, suitably at least 2 GPa, preferably at least 3 GPa, more preferably at least 3.5 GPa. The flexural modulus is preferably in the range 3.5-4.5 GPa, more preferably in the range 3.5-4.1 GPa.

Said surface of said part which includes said array is suitably more or less hydrophobic compared to a surface of the same polymeric material which does not include a said array—i.e. a surface which is preferably substantially smooth. Suitably, said surface which includes said array is more hydrophobic.

The water contact angle of said surface which includes said array of topographical features is suitably at least 88°, preferably at least 90°, more preferably at least 92°, especially at least 94°. The water contact angle may be less than 120°, 110° or 100°. The water contact angle may be assessed as described in Example 3.

In one embodiment, said surface of said part may be treated to increase its hydrophilicity.

This may involve treating a surface which includes a said array with a chemical means, for example an oxygen plasma treatment, to render it more hydrophilic. Preferably, however, said surface of said part is defined substantially entirely by a moulding process and/or the surface is not chemically modified after moulding.

Said part preferably includes at least 60 wt %, 70 wt %, 80 wt %, 90 wt % or 95 wt % of said polymeric material. More preferably, said part consists essentially of said polymeric material. If said part includes material in addition to said polymeric material, it may include less than 10 wt %, preferably less than 5 wt % of other material, for example X-ray contrast material, such a barium sulphate.

Said part is preferably a part of a medical device, for example an implantable medical device. Said device may comprise a device for cardiology or for neuromodulation.

Said array of topographical features is preferably defined by said polymeric material, for example by injection moulding of said polymeric material. A surface of said part which include said array is preferably defined by said polymeric material.

In some cases, said part may be provided with a coating (and suitably the array improves the adhesion of the coating relative to a part in the absence of such an array). Thus, the invention extends to a said part, wherein said topographical features, suitably defined by said polymeric material, are covered by a coating. The surface defined by the coating may be smoother than that of said array and/or may not reproduce the topographical features. In other embodiments, when the coating is applied to topographical features which comprise projections, the coating suitably has a thickness which is less that the height of the projections, so as not to completely cover the projections.

According to a second aspect of the invention, there is provided a method of making a part according to the first aspect, the method comprising the steps of:

(a) selecting a polymeric material according to said first aspect;

(b) injection moulding said polymeric material to define said part, wherein a mould used in said injection moulding is arranged to define topographical features in the moulded part produced.

Said mould suitably includes means for defining topographical features as described according to the first aspect. Thus, said mould suitably includes means for defining an array of topographical features having a maximum dimension which is less than 3 μm and separated by a distance which is less than 10 μm.

The method may comprise designing a notional symmetrical lattice, applying a degree of mis-order to the notional symmetrical lattice by requiring that the centre of each topographical feature is up to one third of C (as defined according to the first aspect) from its respective notional lattice point, thereby designing a mis-ordered lattice, and manufacturing the topographical features according to the mis-ordered lattice.

Preferably, the degree of mis-order is applied to each notional lattice point by a calculation step in which a random number is generated and used to provide one or more displacement amounts to said notional lattice point. For example, for each lattice point of a rectangular or square lattice, a random displacement along one axis may be applied, followed by a random displacement along an orthogonal axis. For a non-orthogonal lattice (e.g. a parallelogram lattice, hexagonal lattice or trigonal lattice), these random displacements may be made along axes of the lattice, or along orthogonal axes. Typically, the random number generated is operated on using a multiplier, that multiplier corresponding to the fraction of C corresponding to the desired maximum mis-order of the array of topographical features.

Preferably, the method comprises the step of forming an array of topographical features using electron beam lithography. This array may be formed on the surface of a master substrate.

The master substrate may be used to create an intermediate substrate. For example, the intermediate substrate may be formed to provide the “negative” topographical features to those of the master substrate. The intermediate substrate may then be used to create the part by securing it in a mould and injecting said polymeric material into the mould so the part is formed with topographical features, corresponding to those on the intermediate substrate.

The intermediate substrate may comprise a silicon substrate or a nickel shim.

It has been found that polymeric materials described herein, for example polyetheretherketone, can advantageously be used as said polymeric material and said topographical features can unexpectedly be satisfactorily reproduced. Suitably, the melt viscosity (MV) of said polymeric material, suitably assessed as described herein, is less than 0.4 kNsm⁻², preferably less than 0.3 kNsm⁻² and, more preferably, less than 0.2 kNsm⁻². The MV is suitably 0.9 kNsm⁻² or greater, suitably 0.11 kNsm⁻² or greater. Preferably, the MV is in the range 0.12 kNsm⁻² to 0.18 kNsm⁻². The part which incorporates said topographical features suitably comprises at least 80 wt %, preferably at least 90 wt %, more preferably at least 95 wt %, especially at least 99 wt % of polyetheretherketone (suitably a polymeric material of formula (XX) wherein t1=0, v1=0 and w1=0).

Said part described herein may be for an implantable medical device. It may be a bone repair device which is suitably arranged to replace or augment bone within the body. For example, it may comprise a device for orthopaedic use (e.g. hip, knee), spinal use, or for maxillofacial use. Thus, said medical device may be an orthopaedic, spinal or maxillofacial device.

Advantageously, the method may be used to produce parts having predetermined and reproducible arrays of topographical features and a multiplicity of substantially identical parts (particularly in terms of substantial identity of said topographical features) may be produced in a rapid manner. Thus, in a third aspect, there is provided a collection comprising a plurality, preferably at least 5, more preferably at least 10, parts according to said first aspect, wherein each part in said collection is substantially identical to each other part. In particular, the topographical features or each part may be substantially identically reproduced on each part in said collection.

According to a fourth aspect of the invention, there is provided a method of treating a human or animal body, the method comprising:

(i) selecting a part according to the first aspect;

(ii) optionally, assembling said part with other parts to produce a device; and

(iii) implanting said part or said device into a human or animal body (especially a human body).

According to a fifth aspect, there is provided use of a medical device as described herein in implant surgery.

Any feature of any aspect of any invention or embodiment described herein may be combined with any feature of any aspect of any other invention or embodiment described herein mutatis mutandis.

Specific embodiments of the invention will now be described, by way of example, with reference to the accompanying drawings, in which:

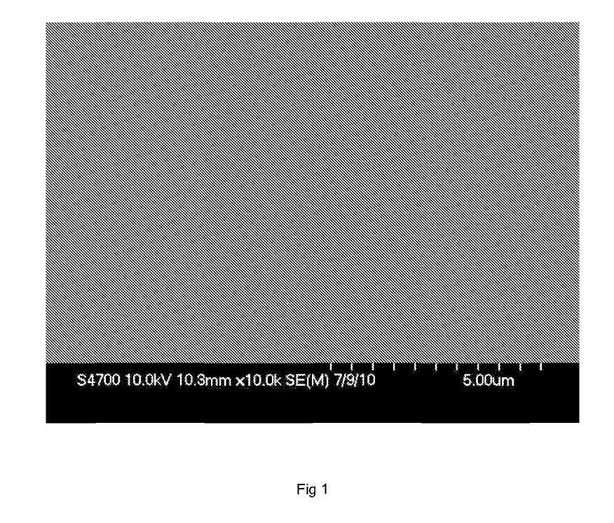

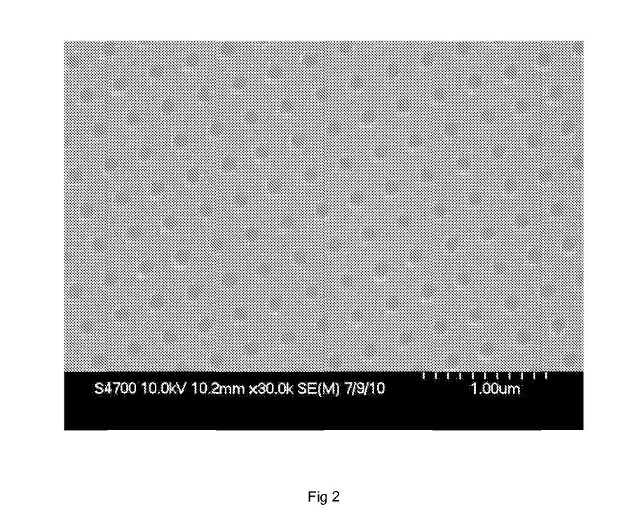

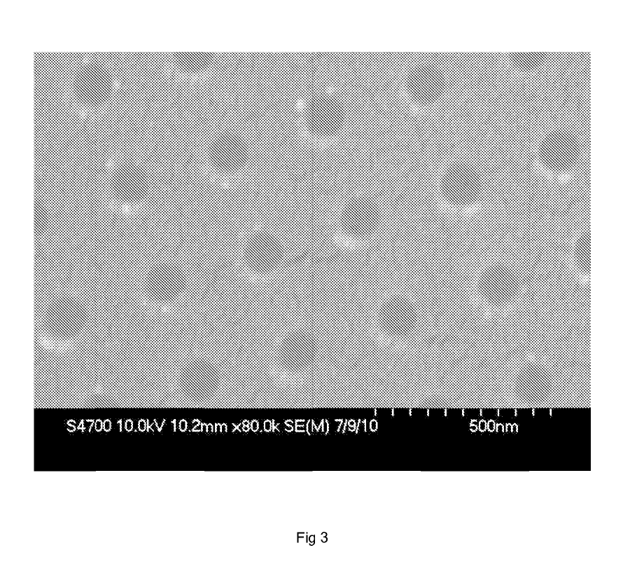

FIGS. 1 to 3 are micrographs of PEEK substrates having nanoscale topographies.

The following material is referred to herein:

PEEK OPTIMA LT3—polyetheretherketone having a melt viscosity of 0.15 kNsm⁻² obtained from Invibio Limited, UK.

PEEK OPTIMA LT1—polyetheretherketone having a melt viscosity of 0.45 kNsm⁻².

In general terms, it has been found that nanoscale topography can be transferred to polyetheretherketone, on an industrial scale, by injection moulding relatively low viscosity PEEK, using a mould in which is arranged a master structure which carries the desired topography. Surprisingly, the nanoscale topography can be reproduced on the PEEK and such a modified surface has been shown to influence cell attachment and differentiation. Further details on the process are produced below.

### EXAMPLE 1

### Manufacture of Substrate Having Predetermined Topographic Features

A suitable pattern having a desired degree of mis-order was produced in a silicon substrate to define a master. This master was formed of silicon since patterning of silicon is well-understood. The silicon master was near atomically flat before patterning and was sufficiently conducting during the electron exposure to avoid sample charging. The desired pattern was generated by a computer program (Matlab followed by WAM) in which a suitable notional lattice was defined and each topographic feature was randomly displaced along the axes of the lattice by a random value. A design pattern typically consisted of 100 nm diameter features separated with an average spacing of 300 nm. Each of the features was randomly off-set with a value of +/−50 nm. The software generates a file suitable for an electron beam lithography tool to read and execute. The silicon substrate was coated with a resist comprising a polymeric material, (typically PMMA was used although other positive and negative resists could also be used) which was susceptible to electron exposure. An electron beam lithography tool (Leica EBPG5-HR100 operating at 50 kV with an 80 nm spot size) generated the pattern onto the flat silicon substrate. The resolution of this specific machine is 5 nm with a similar grid resolution. The stochastic displacement as a result of signal noise, temperature variations was measured to be less than 2 nm. After electron beam exposure, the sample was developed in a mixture of IPA and MIBK at a ratio of 2.5 to 1, respectively, for 30 seconds. This was followed by a rinse with copious amounts of IPA and then blown dry in a stream of nitrogen. This completes the fabrication of the silicon master substrate.

### EXAMPLE 2

### Manufacture of PEEK With Predetermined Topographical Features

A nickel shim (or in some cases more than one may be used) with formed topographical patterns made as described in Example 1 was selected and inserted within an injection moulding machine (ENGEL Victory 80/28). A tool that created plaques of 25 mm×25 mm×2 mm was used. A standard reciprocating screw injection moulding machine was used, with a melt temperature of 380° C. and mould surface temperature in the range 175° C. to 205° C. which was found to give good mould filling and a high level of crystallinity (typically 30%) within mouldings. Injection pressures of 70 to 140 MPa were initially used with holding pressures of 40 to 100 MPa. To create a homogenous melt to aid consistency of shot size a nominal back pressure of 3 MPa was used. A screw speed of 50 to 100 rpm was found to be optimum.

PEEK-OPTIMA LT3 was used as the polymer. 3 mm sized PEEK-OPTIMA granules were pre-dried to a level of less than 0.1 wt % water prior to moulding using an air circulating oven at 150° C. for 3 hours.

Examples of nanoscale topographies produced using the methods described are provided in FIGS. 1 to 3.

### EXAMPLE 3

### Assessment of Surfaces Produced—Contact Angle

This may be assessed by a static sessile drop method using a contact angle goniometer which includes an optical sub-system to capture the profile of a pure liquid on a solid substrate. The angle formed between the liquid/solid interface and the liquid/vapour interface is the contact angle. Current generation systems utilize high resolution cameras and software to capture and analyze the contact angle. Most commonly water is used as the liquid for the measurement and thus the results are generally referred to as water contact angle measurements. Alternatively captive bubble contact angle of air measured under water can be used to assess wettability in a similar fashion.

It was found that a flat PEEK surface produced by injection moulding PEEK into a tool having a substantially flat surface (i.e. the tool did not incorporate topographical features as described) had a contact angle of 85°, wherein a PEEK surface prepared as described herein had a contact angle of 95°. In both cases water was the liquid used.

As an alternative to use of PEEK OPTIMA LT3, PEEK OPTIMA LT1 may be used.

The provision of topographical features as described may have a range of applications as described below.

1. A neurological sensor, implanted as a deep brain stimulator. Problem to solve is that these sensors become covered with soft tissue that interferes with the signal. Preventing soft tissue attachment would be beneficial.

2. A bearing surface with a nanotopography mimicking cartilage, so that contact of the fluid film with the bearing surface is altered to benefit the bearing between two surfaces.

3. The backside of an orthopaedic device eg. femoral knee component that requires better bone attachment.

4. The backside of an orthopaedic device eg. hip cup that requires coating with HA; nanotopography could facilitate the adhesion of the coating.

5. A medical device eg. spinal implant cage may include topographical features which facilitate better bonding with bone.

6. A CMF device where zones of patterns may be defined so that enhanced bone contact is achieved and/or additional or separate zones may be defined where soft tissue attachment is desired.

7. A CMF device eg. mid face implant orbital socket where attachment of soft tissue may not be wanted, to facilitate gliding operation of muscles and tendons.

8. An industrial PEEK device that requires coating with a paint.

9. An industrial or medical device that requires unique identification to act as a security marker. These parts could be moulded with nanotopography. The pattern would be invisible to the naked eye but act as a means of security detection when viewed under a microscope or other magnifying machine.

10. A implantable AIMDs device such as a pacemaker or pain limiting device. PEEK receptacles containing electronics or sensors or batteries or parts susceptible to fluid ingress may have inside or outside surfaces patterned to prevent fluid ingress by creating a hydrophobic barrier.

11. A marketing device eg. where parts made by one company are required to be differentiated from those of another company.

12. Parts that may need the impression of a different colour or surface texture when visualised. When a part is viewed from a different angle it may be given another visual property compared to a part that is not patterned.

13. A device that is required to be water repellent.

14. A part or device that is required to prevent bacterial attachment, such as a catheter or implant (cardiovascular, spine, orthopaedic, trauma, dental).

15. A device is used in several environments and requires varying properties in each environment. For example, an industrial application may desire a device which requires attachment of another material or bonding in one zone and requires repulsion of fluids at another zone. A medical device such as a dental device may require bone bonding in one zone, whilst requiring soft tissue attachment at the gingival zone, and then adhesion of cements in another zone.

16. A part which is subsequently over moulded and requires a better interfacial bond.

17. A part or device that may be subsequently subjected to additional enhancing processes such as RF oxygen, ammonia or nitrogen or other plasma.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A part which comprises a polymeric material, wherein a surface of said part includes an array of topographical features which comprise spaced apart projections or recesses, wherein said topographical features have a maximum dimension which is less than 3 μm and said topographical features are separated by a distance which is less than 10 μm, wherein said polymeric material has a moiety of formula 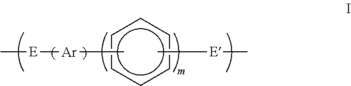
and/or a moiety of formula 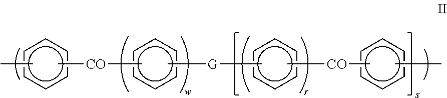
and/or a moiety of formula 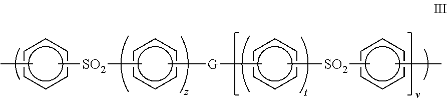
wherein m, r, s, t, v, w and z independently represent zero or a positive integer, E and E′ independently represent an oxygen or a sulphur atom or a direct link, G represents an oxygen or sulphur atom, a direct link or a —O-Ph-O— moiety where Ph represents a phenyl group and Ar is selected from one of the following moieties (i)**, (i) to (iv) which is bonded via one or more of its phenyl moieties to adjacent moieties

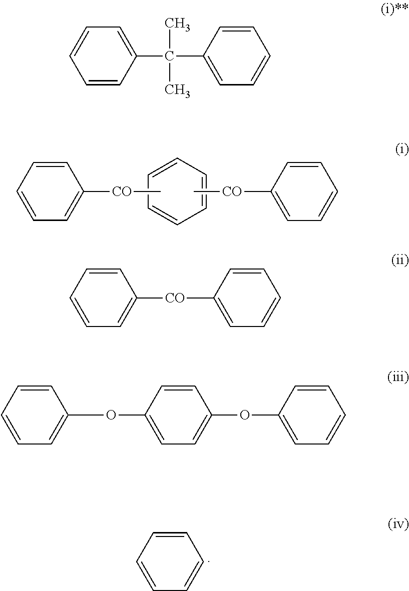

2. A part according to claim 1, wherein said array of topographical features include at least 100,000 of said topographical features per mm².

3. A part according to claim 1, wherein when said topographical features comprise projections, the maximum height of said projections is less than 10 μm and when said topographical features comprise recesses, the maximum depth of the recesses is less than 10 μm.

4. A part according to claim 1, wherein said topographical features are circular, triangular or square in plan view.

5. A part according to claim 1, wherein said respective topographical features have a maximum area of less than 20 μm².

6. A part according to claim 1, wherein the minimum area of said respective topographical features is at least 200 nm².

7. A part according to claim 1, said device having an arrangement of topographical features arrayed in a pattern based on a notional symmetrical lattice in which the distance between nearest neighbour notional lattice points is C and is between 10 nm and 10 μm, and wherein the topographical features are locally mis-ordered such that the centre of each topographical feature is a distance of up to one half of C from its respective notional lattice point.

8. A part according to claim 5, wherein C is at least 20 nm and is at most 9 μm.

9. A part according to claim 7, wherein the height or depth of the topographical features is at least 5% of C and the diameter of the topographical features is at least 10% of C.

10. A part according to claim 1, wherein said polymeric material is a homopolymer having a repeat unit of general formula 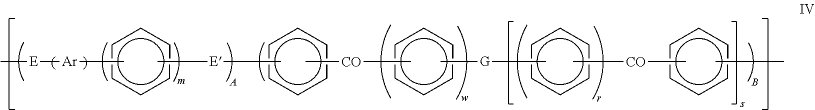
or a homopolymer having a repeat unit of general formula 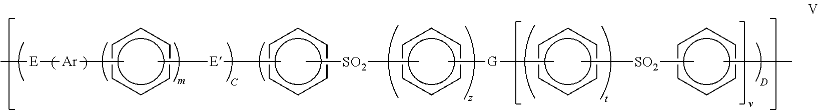
or a random or block copolymer of at least two different units of IV and/or V, wherein A, B, C and D independently represent 0 or 1 and E, E′, G, Ar, m, r, s, t, v, w and z are as described in any preceding claim.

11. A part according to claim 1, wherein said polymeric material comprises a repeat unit of formula (XX) 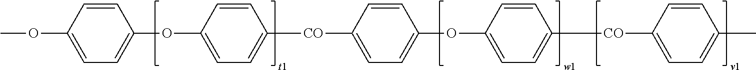
where t1, and w1 independently represent 0 or 1 and v1 represents 0, 1 or 2.

12. A part according to claim 11, wherein t1=1, v1=0 and w1=0.

13. A part according to claim 1, wherein said polymeric material has a MV of less than 0.5 kNsm⁻².

14. A part according to claim 1, wherein the water contact angle of said surface which includes said array of topographical features is at least 88° and is less than 120°.

15. A part according to claim 1, wherein said surface of said part is treated to increase its hydrophilicity.

16. A part according to claim 1, wherein said part is of a medical device.

17. A part according to claim 16, wherein said device is for cardiology or for neuromodulation.

18. A part according to claim 1 which is one part of a collection of parts which includes at least five substantially identical parts.

19. A method of making a part according to claim 1, the method comprising the steps of:
(a) selecting a polymeric material according to claim 1;
(b) injection moulding said polymeric material to define said part, wherein a mould used in said injection moulding is arranged to define topographical features in the moulded part produced.

20. A method of treating a human or animal body, the method comprising:
(i) selecting a part according to claim 1;
(ii) optionally, assembling said part with other parts to produce a device; and
(iii) implanting said part or said device into a human or animal body.
